(19) United States
(12) Patent Application Publication
(10) Pub. No.: US 2004/0137458 A1
(43) Pub. Date: Jul. 15, 2004

(54) Molecular tag code for monitoring a product and process using same

(76) Inventors: Christian Archambault, Laval (CA); Daniel Plante, Laval (CA)

Correspondence Address: Goudreau Gage Dubuc, Stock Exchange Tower Suite 3400, P O Box 242, 800 Place Victoria, Montreal, QC, H4Z 1E9, CA

(21) Appl. No.: 10/476,373

(22) PCT Filed: May 3, 2002
(86) PCT No.: PCT/CA02/00678

(51) Int. Cl. C12Q 1/68; C07H 21/04

(52) U.S. Cl. 435/6; 536/24.3

(57) ABSTRACT

In a first aspect of the invention, there is provided a nucleic acid tag comprising: a single-stranded nucleic acid sequence portion having a 5′ end portion and a 3′ end portion; at least two amplification primer binding sequences in said 5′ end and 3′ end portions; internal to these primer binding sequences, at least one marker of about 18 to about 25 nucleotides; and between these markers, a spacer, wherein the spacer has a length sufficient to allow molecular beacons to properly attach to amplification copies of the marker sequences bordering the amplification copy of the spacer and wherein the nucleic acid sequences of primer binding sequences, the marker and the spacer are chosen so as to minimize or prevent secondary structure formation. The said 5′ end and 3′ end portions are preferably protected from degradation. This molecular tag is simple and inexpensive to produce and easy to detect. There is also provided methods of identifying 15 substances with same and methods of detecting same in a substance. In a second aspect of the invention, there is also provided a use of a molecular tag for characterizing qualitatively and/or quantitatively at least one procedure of a manufacturing process for manufacturing an end product from at least one raw and/or intermediate product.

### FIELD OF THE INVENTION

In a first aspect, the present invention relates to a molecular barcode for monitoring products and/or processes using same and/or processes for detecting same. In particular, the present invention relates to a molecular barcode for monitoring, detecting and tracing substances or goods used in manufacture or released into trade or environment and methods for monitoring, detecting and tracing substances or goods using these molecules. In a second related aspect, the present invention relates to a molecular barcode for use in quality control applications.

### BACKGROUND OF THE INVENTION

It is often desirable to tag articles of manufacture destined to trade to permit their easier identification down the stream of trade. It is often required to assess the authenticity of a number of objects which may derive their value from their origin (such as works of art and other collectibles). Other objects that are advantageously tagged include identification documents such as passports, wills, stock certificates, visas, credit cards, electronic equipment, designer clothes, perfumes and any other product that may be the subject of counterfeiting. Any good that could be the subject of theft (household appliances, televisions, tapes, compact disks, cars, etc.) may also profitably be tagged.

Further, some goods may be destined to limited channels of trade and their diversion to other channels could be illegal or unauthorized. For instance, certain types of goods may be prohibited in certain jurisdictions and/or their importation may be restricted. In addition, the right to sale certain goods in certain jurisdictions or channels of trade may be exclusively granted to certain person so that their sale in these restricted channels of trade by third parties could constitute breaches of contracts. Since these goods are authentic, it may be difficult to asses whether they have been improperly used or sidetracked from their legitimate channels of trade and/or jurisdictions.

In addition, to be able to identify counterfeited products or stolen goods, it may be desirable to tag goods or substances to be able to determine their origin when they have been improperly used. It may also be useful to simply be able to monitor the distribution of certain goods. Such goods or substances include natural resources such as water, minerals, plants and animals; commercial by-products such as pollutants; chemicals such as drugs, explosives and manufactured product such as guns and food stuff, or particular steps of manufacture or modifications brought to a good during its manufacture.

As an illustration of these last applications, it may be useful to be able to track the specific company responsible for the pollution of a river or identify the retailer of the weapon used to commit a crime.

For these purposes, goods have traditionally been tagged with marks such as serial numbers and bar codes. These and other traditional tagging devices may easily be removed from the stolen or otherwise improperly used product or be copied and affixed to counterfeited goods.

A number of tagging techniques were suggested that limited the possibility to remove or reproduce the tags. Invisible markers such as for instance, infrared or ultraviolet dyes and biological markers that included proteins, amino acids, nucleic acids, polypeptides, hormones and antibodies were proposed. Nucleic acid tags in particular provide a number of significant advantages. Firstly, the identity of the nucleic acid tags being based on their sequence can be known only to their legitimate users. Tags made of nucleic acids are virtually undetectable and are impossible to duplicate without prior knowledge of the nucleic acid sequence. For this reason the level of protection that they provide against counterfeiting is high. In addition, an extremely large number of tags can be made by using different combinations of bases. One owner could therefore use different tags or tags having more than one target nucleic acid sequence to identify each of its product and also its various products lots and dates of production.

U.S. Pat. No. 6,030,657 discloses a tagging technique using encapsulated nucleic acid as tags, in association with an agent that emits detectable wavelengths of energy. Various encapsulants are suggested: casein, and spore-forming bacteria to prevent degradation of the nucleic acids. However, these tags seem to be detectable by third parties since it is suggested to use junk DNA to mask the tags.

U.S. Pat. No. 5,451,505 also discloses a nucleic acid labeling technique that uses varying amounts of nucleic acid bound or not to the product being tagged. The examples disclosed in this patent show that the concentration of nucleic acid used varies according to the product tagged: 16,000 pg/g gun powder, 22,000,000 pg/ml oil, 7,400,000 pg/tablet medicines and 20,000 pg/g food. U.S. Pat. No. 5,451,505 discloses nucleic acid molecules tags comprising at least 20 bases to avoid false results due to contamination and less than 1000 bases are preferred for their greater stability against degradation. According to the compositions disclosed therein, certain chemically active substances such as foodstuffs with enzymatic activity or acidic pharmaceuticals may require that a protective composition be added to the nucleic acid tag to avoid their degradation. Suggested protective compositions include encapsulants such as liposomes, detergents for non-polar liquid substances such as oils. Preferred nucleic acid tags used are double stranded DNAs.

Providing tags with encapsulants or other protective compositions can be time consuming and expensive. Further, the use of encapsulants may increase the toxicity of the tags. The use of double stranded (ds) molecules as tags as in U.S. Pat. No. 5,451,505 is more difficult to produce and costly than that of single stranded nucleic acids. Furthermore, ds tags present the risk of recombining into the genome of a living organism which comes in contact with these tags. Further, the methods of detection of nucleic acid tags of the prior art are often time-consuming as they often require an overnight detection procedure.

Efficient large-scale use of molecular tags requires their rapid and easy detection and identification in the tagged material. Traditional methods of detection and identification such as sequencing or Southern or Northern hybridization are not suitable for large scale use because of their complexity and of their lengthy procedure.

There thus remains a need to provide a nucleic acid tag which overcomes the drawbacks of those of the prior art. More particularly, there remains a need to provide tags having at least one of the following advantages: (1) an increased long term resistance to degradation without the use of encapsulant or synthetic derivatized nucleotides; (2) virtually undetectable without prior knowledge of its presence; and (3) simple and inexpensive to produce.

There also remains a need to provide a nucleic acid tag that can be quickly and easily detected by those who have the prior knowledge of its presence. There also remains a need for a molecular tag optimized for sensitive detection with new high-throughput homogeneous detection methods.

There also remains a need to provide a molecular tag that does not involve the use of potentially hazardous living organisms which could end up in the final tag preparation or of molecules or tags presenting a very low risk of being recombined into the genome of living organisms.

There also remains a need for low cost molecular tags.

Molecular tags of the prior art have been used as substitutes for barcodes: they were used for the tagging of various finished products in order to identify their source. For example, U.S. Pat. No. 6,030,657 discloses the use of encapsulated nucleic acid molecular tags to mark goods for verifying their authenticity and their sale in proper channels of trade.

U.S. Pat. No. 5,451,505 discloses the use of nucleic acid molecular tags which are preferably encapsulated to mark goods to be able to trace them in the environment in the stream of trade and be able to identify their source.

U.S. Pat. No. 5,981,283 used molecular tags to enable a tracing of various liquids such as gasoline to determine whether they have been improperly diluted. However, this patent is concerned with a chemical tagging molecule broadly taught as containing C, H, N, O and S and wherein a detection of the chemical tag is dependent on mass spectrum analysis.

There thus remains a need for more versatile molecular tags for use for example in identifying not only finished product but also raw products and to monitor the production of a particular product. For instance, it is often desirable to be able to determine quickly and efficiently whether the proper percentage of various components are present in the end products, or to determine if one or more components were added, in what concentration and the like.

There also remains a need for identifying not only products per se but for determining whether finished, raw or intermediate products have been properly manufactured. There remains a need for molecular tags to mark and monitor processes rather than products.

There thus remains a need for simple and rapid methods of conducting quality control analysis. Many known quality control monitoring methods involve time-consuming and inefficient analyses. There is therefore a need for improved methods of monitoring production and quality controls.

The present invention seeks to meet these and other needs.

The present description refers to a number of documents, the content of which is herein incorporated by reference in their entirety.

### SUMMARY OF THE INVENTION

Generally, the present invention relates to nucleic acid molecular tags and uses thereof which are aimed at overcoming at least some of the drawbacks of the prior art.

In a first aspect, the present invention relates to nucleic acid tags used for monitoring, detecting and tracing goods and substances released in the stream of trade or in the environment.

The present invention provides or use a synthetically-produced nucleic acid tag with increased resistance to degradation that do not require a protective composition such as an encapsulant.

The molecular tags of the present invention may be used at extremely low concentration, thereby greatly limiting risks related to their ingestion when used to label food products. In addition, since in a preferred embodiment the molecular tags of the present invention are single stranded, they overcome the drawbacks associated with double stranded molecules. Furthermore, while the molecular tags of the present invention could be encapsulated, their resistance to degradation may decrease the need for encapsulation.

The present invention aims at providing a rapid detection method, significantly faster than the overnight requirements of some of the methods of the prior art.

As indicated earlier, traditional methods of detection and identification such as sequencing, Southern hybridization, or Northern hybridization are not considered suitable for large scale use of molecular tags because of their complexity and of their lengthy procedure. New tools for detecting and identifying nucleic acids have been devised. These methods combine the steps of amplification and detection of the amplified products into one reaction called “homogenous detection methods”. These technological advances have facilitated simultaneous treatment of a large number of sample.

Specific embodiments of the present invention have taken advantage of these technological advances in methods for detecting and identifying nucleic acids. Specific embodiments of the present invention are particularly suitable for detection by one of these tools called molecular beacons. Using molecular tags adapted for detection by molecular beacons considerably simplifies the manipulations that are otherwise required when traditional detection and identifications means are used.

Use of molecular beacons with traditional molecular tags tends to produce fluorescent signals that are below predicted values. This decreased signal reduces the sensitivity of the detection test and also increases the possibility of misidentifying the tags because of the small difference between specific and non-specific signals. It has now been determined that this problem can be attributed to secondary structures formed randomly in the molecular tags. These secondary structures contribute to shift the thermodynamic equilibrium of molecular beacons to their non-hybridized forms. Careful selection of the sequence of the tag is therefore important to avoid the formation of secondary structures. In a particularly preferred embodiment, sequences of specific regions of the tag are chosen to comprise non-pairing nucleotides exclusively.

Before the present invention, many nucleic acid tags used for food products required to be encapsulated to resist degradation by enzymatic activity or acidic compositions. The present invention provides molecular tags which can be considered as safe, not counterfeitable, and which may be resistant to degradation in numerous environments (e.g. fruit juices, meats, paint) and enable a quick and easy detection.

The molecular tags of the first aspect of the present invention can be advantageously used in minimal amounts to efficiently tag products. According to one embodiment of the present invention, using a molecular tag comprising one marker, the following concentrations were shown to be sufficient: 35 pg/g to tag ground beef and 3,500,000 pg/ml to tag gasoline. As a comparison, U.S. Pat. No. '505 discloses uses of nucleic acid in concentration of 16,000 pg/g to tag gunpowder, 22,000,000 pg/ml to tag oil, 7,400,000 pg/tablet to tag medicines and 20,000 pg/g to tag food. The molecular tags of the present invention can thus be used at significantly reduced concentration. For example, they can be used generally in concentrations of less than 10,000 pg/g of product, and preferably in concentrations of less than 1,000 pg/g of product.

In accordance with one embodiment of the present invention, there is therefore provided a nucleic acid tag for monitoring, detecting or tracing substances, tag comprising (1) a single-stranded nucleic acid region, (2) two ends being capable of pairing with a complementary nucleotide sequence; and (3) at least one marker sequence having a number of non-complementary nucleotides sufficient to minimize or prevent the formation of secondary structure within marker under normal conditions of use.

In accordance with another embodiment of the present invention, there is also provided a nucleic acid tag comprising: (1) a single-stranded nucleic acid sequence portion having a 5′ end portion and a 3′ end portion; (2) at least two amplification primer binding sequences in 5′ end and 3′ end portions; (3) internal to these primer binding sequences, at least one marker of about 18 to about 25 nucleotides; (4) and between these markers, a spacer, wherein the spacer has a length sufficient to allow molecular beacons to properly attach to amplification copies of the marker sequences bordering the amplification copy of the spacer and wherein the nucleic acid sequences of primer binding sequences, the marker and the spacer are chosen so as to minimize or prevent secondary structure formation. The amplification primers are preferably PCR primers. The 5′ end and 3′ end portions are preferably protected. Non-limiting examples of protectors of the end portions include self-complementary sequences or additional nucleotides. The additional nucleotides can be complementary so as to form a stem having preferably a length of 3 to 8 nucleotides.

In accordance with yet another embodiment of the present invention, there is provided a method of tagging a substance for its identification comprising: tagging the substance with a molecular tag of the present invention, releasing the tagged substance in the stream of trade or in the environment; whereby the substance suspected to contain the tag can be identified by subsequent amplification and qualitative and/or quantitative detection of the molecular tag in the substance. In a second aspect of the invention, the present invention relates to molecular tags used for marking processes and quality control applications. In this second aspect, the present invention also relates to marking raw products intended for manufacture and for monitoring the process of manufacture from the raw material to the final product.

The invention also relates to a use of a molecular tag to identify manufacture processes, and monitor it. The invention also relates to a use of a molecular bar code in quality control applications. According to an embodiment of a second aspect of the present invention, there is provided a use of a molecular tag for characterizing qualitatively and/or quantitatively at least one procedure of a manufacturing process for manufacturing an end product from at least one raw and/or intermediate product.

According to another embodiment of the second aspect of the present invention, there is provided a use of a molecular tag for characterizing qualitatively and/or quantitatively at least one procedure of a manufacturing process for manufacturing an end product from at least one raw and/or intermediate product, wherein the at least one procedure is a mixing procedure comprising adding a defined quantity of a specific molecular tag in one of the raw and/or intermediate products, prior to mixture with at least one other raw and/or intermediate product, to obtain a tagged product; mixing the tagged product with the at least one other raw and/or intermediate product to obtain a mixture; and determining the quantity of molecular tag in the mixture, whereby the quantity of tagged product in the mixture can be deduced from the quantity of molecular tag contained in the mixture.

According to another embodiment of the second aspect of the present invention, there is provided a method of identifying a defective production line in a manufacturing process which comprises a pooling of pre-manufactured products comprising adding a specific molecular tag in at least one of the pre-manufactured product or in a manufactured product in each production line prior to a pooling together of the manufactured products; identifying defective manufactured products; identifying the molecular tag in the defecting products, whereby the identity of the molecular tag in the defecting products leads to the identification of the defective production line.

Molecular tags as used herein are meant to include tags consisting of nucleic acids such as DNA, RNA or DNA-RNA chimeras, nucleotide sequences comprising synthetic nucleotides analogs designed to be more resistant; inorganic phosphor compositions, light wave emitting substances, hydrocarbons and any other molecular tag that can appropriately be used to tag products. In particular, the expression molecular tags are meant to include the tags described in U.S. Pat. No. 5,451,505, U.S. Pat. No. 6,030,657; U.S. Pat. No. 6,153,389; U.S. Pat. No. 6,172,218; U.S. Pat. No. 5,981,283. It will be understood by one of ordinary skill in the art that molecular tags intended to mark products of the present invention or products manufacturing processes should not present risks for the health of those for which the products are intended. In non limiting examples, these products are foodstuff products or foodstuff manufacturing processes. The person of ordinary skill will know the characteristics that these molecular tags should have and will thus be preferably chosen to be considered innocuous for the user.

Any nucleic acid may be used and are encompassed as molecular tags according to the present invention. However, single stranded DNA are preferred: (1) ssDNA is the easiest and cheapest nucleic acid to synthesize in vitro; (2) its synthesis does not involve the use of potentially hazardous living organisms, for example bacteria (such as would be required if one were to use plasmids as taggant molecule) which could end up in the final taggant preparation; and (3) the risk of recombination of ssDNA molecule into the genome of living organisms is very low (much lower than if dsDNA were used). This last consideration is significant if the nucleic acid tags are to be ingested or released in the environment. Thus, in a preferred aspect, the molecular tag in accordance with the present invention is comprised mostly of ssDNA and hence should not suffer from the drawbacks related to genetically modified organisms (GMDs).

Many suitable detection methods are encompassed herein in order to detect the molecular tag in accordance with the present invention. For instance, when the molecular tag is a nucleic acid, the following non-limiting methods of amplification thereof are suitable: polymerase chain reaction (PCR); rolling circle amplification (RCA); signal mediated amplification of RNA technology (SMART); split complex amplification reaction (SCAR); split promoter amplification of RNA (SPAR).

When the method to amplify DNA used is PCR, non-limiting examples of suitable methods to detect the presence of PCR products include the followings: agarose or polyacrylamide gel, addition of DNA labeling dye in PCR reaction (e.g. ethidium bromide, picogreen, etc.) and detection with suitable apparatus (e.g. fluorometer or real-time PCR apparatus).

Similarly, when PCR is used to amplify the tags according to the present invention, non-limiting suitable methods to determine the sequence of PCR products include sequencing reaction (either manual or automated); restriction analysis (e.g. when restriction sites were built into the tag's sequences), or any method involving hybridization with a sequence specific probe (e.g. Southern or Northern blot, TaqMan™ probes, molecular beacons, Scorpions probes and the like). Of course, as will be seen below, other amplification methods are encompassed by the present invention. In particular, methods incorporating molecular beacons are the preferred methods for detecting the molecular tags according to one aspect of the present invention.

The nucleic acid tags of the present invention encompass DNA sequences, RNA sequences or chimeras thereof. According to a preferred embodiment, the nucleic acid tags of the present invention are DNA sequences while in a preferred embodiment, the molecular tags of the present invention are resistant to degradation. The introduction therein of nucleotides which are more resistant to degradation could further improve their stability.

In order to provide a clear and consistent understanding of terms used in the present description, a number of definitions are provided hereinbelow.

The terms “molecular barcode” and “molecular tag” are used herein interchangeably and refer to the nucleic acid molecules of the present invention.

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

Unless defined otherwise, the scientific and technological terms and nomenclature used herein have the same meaning as commonly understood by a person of ordinary skill to which this invention pertains. Generally, the procedures for molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, New York).

As used herein, “nucleic acid molecule”, refers to a polymer of nucleotides. Non-limiting examples thereof include DNA (e.g. genomic DNA, cDNA), preferably synthetic DNA, RNA molecules (e.g. mRNA), preferably synthetic RNA and chimeras thereof. The nucleic acid molecule can be obtained by cloning techniques or synthesized. The nucleic acids can be double-stranded or single-stranded (coding strand or non-coding strand [antisense]). Preferably, the nucleic acid is single-stranded and more preferably it is comprised of at least a majority of deoxynucleotides.

While the term “single-stranded” is very well-known in the art, it should be understood that a single-stranded nucleic acid can, under certain conditions, fold such as to form a secondary or tertiary structure. As will be seen and exemplified below, in one particular embodiment of the present invention, the single-stranded nucleic acid tag of the present invention is comprised of deoxynucleotides and comprises a double-stranded region formed by the hybridization of the 5′ end portion and 3′ end portion thereof. Of course, such double-stranded regions could also be formed using an oligonucleotide which hybridizes to the 5′ or 3′ end. The person of ordinary skill will understand how to design such oligos. As well, the person of ordinary skill will understand that other means of protecting the ends of the molecular tags could also be used (e.g. chemical modification . . . ).

The term “recombinant DNA” as known in the art refers to a DNA molecule resulting from the joining of DNA segments. This is often referred to as genetic engineering. The same is true for “recombinant nucleic acid”.

The term “DNA segment”, is used herein, to refer to a DNA molecule comprising a linear stretch or sequence of nucleotides. This sequence when read in accordance with the genetic code, could encode a linear stretch or sequence of amino acids which can be referred to as a polypeptide, protein, protein fragment and the like.

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

The nucleic acid (e.g. DNA, RNA or chimeras thereof) for practicing the present invention may be obtained according to well-known methods.

Oligonucleotide probes or primers used in the present invention are at least 10 nucleotides in length, preferably between 15 and 25 nucleotides, and they may be adapted to be especially suited to a chosen nucleic acid amplification system. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The term “DNA” molecule or sequence (as well as sometimes the term “oligonucleotide”) refers to a molecule comprised of the deoxyribonucleotides adenine (A), guanine (G), thymine (T) and/or cytosine (C) and/or any analog to these nucleotides (analogs and modified nucleotides are well known in the art; examples thereof can be found in section 116 of the Rules of the Canadian Patent Act), in a double-stranded or preferably in a single-stranded form. When in a double-stranded form, it could, if desired, comprise or include a “regulatory element”.

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989, supra) and are commonly known in the art.

As alluded to earlier, hybridization can also occur in solution and be responsible for generating a double-stranded region (intra- or inter-molecular) of at least a part of the molecular tag of the present invention.

Probes of the invention can be utilized with naturally occurring sugar-phosphate backbones as well as modified backbones including phosphorothioates, dithionates, alkyl phosphonates and α-nucleotides and the like. Modified sugar-phosphate backbones are generally taught by Miller, 1988, Ann. Reports Med. Chem. 23:295 and Moran et al., 1987, Nucleic Acids Res., 14:5019. Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA), Northern blots (RNA detection) and homogeneous detection methods. Although less preferred, labeled proteins and antibodies could also be used to detect a particular nucleic acid sequence to which it binds. Other detection methods include kits containing probes on a dipstick setup and the like. Of course, it might be preferable to use a detection method which is amenable to automation. A non-limiting example thereof includes a chip comprising an array of different probes.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well-known methods (Sambrook et al., 1989, supra). Non-limiting examples of labels include ³H, ¹⁴C, ³²P, and ³⁵S. Non-limiting examples of detectable markers include ligands, fluorophores, chemiluminescent agents, enzymes, antibodies, molecular beacons, TaqMan™, Scorpions™ and the likes. Other detectable markers for use with probes, which can enable an increase in sensitivity of the method of the invention, include biotin and radionucleotides. It will become apparent to the person of ordinary skill that the choice of a particular label dictates the manner in which it is bound to the probe.

As commonly known, radioactive nucleotides can be incorporated into probes for detecting amplification products according to methods of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (e.g. uniformly labeled DNA probe using random oligonucleotide primers in low-melt gels), using the SP6/T7 system to transcribe a DNA segment in the presence of one or more radioactive NTP, and the like.

As used herein, “oligonucleotides” or “oligos” define a molecule having two or more nucleotides (ribo- or deoxy-ribonucleotides or both). The size of the oligo will be dictated by the particular situation and ultimately on the particular use thereof and adapted accordingly by the person of ordinary skill. An oligonucleotide can be synthesized chemically or derived by cloning according to well-known methods.

As used herein, a “primer” defines an oligonucleotide which is capable of annealing to a target sequence, thereby creating a double stranded region which can serve as an initiation point for DNA synthesis under suitable conditions. In one embodiment, the primer could also protect the ends of the molecular tag.

Amplification of a selected, or target, nucleic acid sequence may be carded out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of which are incorporated herein by reference). In general, PCR involves a treatment of a nucleic acid sample (e.g., in the presence of a heat stable DNA polymerase) under hybridizing conditions, with one oligonucleotide primer for each strand of the specific sequence to be detected. An extension product synthesized from each primer is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following EtBr staining of the DNA following gel electrophoreses, or using a detectable label in accordance with known techniques, and the like. For a review on PCR techniques, see PCR Protocols, A Guide to Methods and Amplifications, Michael et al. Eds, Acad. Press, 1990. Detection methods using molecular beacons are preferred according to the present invention.

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

Molecular tags according to the present invention can be derived from the nucleic acid sequences and modified in accordance to well known methods. For example, some molecular tags can be designed to be more resistant to degradation to the various products to which they may be added by using, for example, nucleotide analogs and/or substituting chosen chemical substituents thereof, as commonly known in the art.

The present invention also relates to a kit comprising the molecular tag of the present invention, and comprising at least one primer which is specific to at least one marker sequence on the tag and suitable buffers and reagents. Thus, the present invention also relates to kits for detecting at least one molecular tag in a sample, comprising nucleic acid primers and a probe, such as a molecular beacon specific to the molecular tag marker in accordance with the present invention. For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample (e.g. fruit juice, fuel, meat, or purified nucleic acid), a container which contains the primers which are specific to primer binding sites of the molecular tags, containers which contain heat stable enzymes, such as TAQ, containers which contain wash reagents, and containers which contain the reagents used to detect and/or quantify the extension products, preferably the molecular beacons that are specific to the molecular tag markers enabling the identification of the tagged product.

### BRIEF DESCRIPTION OF THE DRAWINGS

Having thus generally described the invention, reference will now be made to the accompanying drawings, showing by way of illustration a preferred embodiment thereof, and in which:

FIG. 1 shows the secondary structure of an embodiment of the nucleic acid tag of the present invention.

FIG. 2 schematically illustrates different sections of an embodiment of the nucleic acid tag of the present invention;

FIG. 3 is a graphic illustrating the detection of one embodiment of the molecular tag of the present invention using molecular beacons;

FIG. 4 is a graphic showing the effect of secondary structures of molecular tags according to one embodiment of the present invention on the fluorescence intensity generated by molecular beacons;

FIG. 5 illustrates the effect of additional nucleotides outside of the PCR primer binding sites using an embodiment of the molecular tag of the present invention on amplification efficiency;

FIG. 6 illustrates the stability of a molecular tag according to one embodiment of the present invention in unleaded gasoline;

FIG. 7 illustrates the stability of a molecular tag according to one embodiment of the present invention in ground beef;

FIG. 8 shows the recovery of a molecular tag according to one embodiment of the present invention using streptavidin-coated magnetic microparticles;

FIG. 9 graphically illustrates real-time PCR detection of a molecular tag according to a specific embodiment of the present invention, namely molecular tag 11.1 with a FAM-labeled molecular beacon;

FIG. 10 graphically illustrates real-time PCR detection of a molecular tag according to a specific embodiment of the present invention, namely molecular tag 9.8 with Texas-Red-labeled molecular beacon;

FIG. 11 graphically illustrates multiplex real-time PCR detection of two molecular tags according to specific embodiments of the present invention, namely molecular tags 11.1 and 9.8; and

FIG. 12 illustrates the use of a biotin/streptavidin method of extracting the molecular tags of the present invention of tagged products. In particular, panel A schematically shows the structure of a biotin-labeled molecular tag according to a specific embodiment of the present invention; panel B illustrates the biotin-labeled molecular tags captured by streptavidin-coated magnetic microparticles;

FIG. 13 graphically illustrates the fluorescence produced as a function of the number of tags in the PCR reaction.

Other objects, advantages and features of the present invention will become more apparent upon reading of the following non-restrictive description of preferred embodiments with reference to the accompanying drawing which is exemplary and should not be interpreted as limiting the scope of the present invention.

### DESCRIPTION OF PREFERRED EMBODIMENTS

First Aspect of the Invention

The present invention concerns nucleic acid fragments preferably DNA fragments that are added (mixed into liquid products or sprayed or otherwise deposited on solid products) to the product or substances to be tagged. Varying their internal nucleotides sequence can produce unique nucleic acid tags.

For identification, the nucleic acids are first extracted and purified, if necessary, from the tagged product and amplified by PCR. The particular nucleotide sequence of the tag is determined by hybridization with a set of complementary probes using a technology referred to as [symbol] molecular beacons [symbol] . Molecular beacons are short oligonucleotide probes that emit fluorescence when they are bound to their complementary target. In the absence of their target, the molecular beacons are dark. Amplification products of the molecular tags according to the first aspect of the present invention are preferably detected with molecular beacons as described in U.S. Pat. No. 5,925,517. Molecular beacons as described in U.S. Pat. Nos. 6,037,130; 6,103,476; and 6,150,097 can also preferably be used as detection probes for the present invention: they may rapidly detect amplification products and require fewer manipulations than traditional detection tools.

For end-point detection, molecular beacons can be added at the end of the amplification or to the PCR tube prior to amplification.

When molecular beacons are added after amplification, the following procedure may be followed. They are pre-distributed in the wells of the plate, along with a suitable buffer (generally 1×PCR buffer supplemented with MgCl₂ at a final concentration of 4 mM). The concentration of each molecular beacon is comprised between 0.1 and 1 mM. The particular concentration of each molecular beacon needed depend on the intrinsic thermodynamics of each beacon, and these vary according to their particular DNA sequence. The particular fluorophore used also has an impact on the final concentration of the beacon. One skilled in the art is able to determine the concentration needed for each molecular beacons by considering the appropriate parameters (Tyagi et al., 1996, “Molecular beacons: probes that fluoresce upon hybridization” 14 Nature Biotechnol. 303-308). The molecular beacons are distributed in a known pattern on the plate, and each beacon is specific for the sequence of one marker. In one particular embodiment are combined 3 molecular beacons in the same well when each beacon is coupled to a different fluorophore. After mixing of the solutions and PCR products, the molecular beacons for which the complementary marker is present becomes fluorescent. Since the specificity and position of each molecular beacon on the plate is known, analysis of the fluorescence pattern reveals the identity of the nucleic acid tag present in the analyzed sample.

More preferably however, molecular beacons are added to the PCR tubes prior to the amplification. After amplification, the amplification products are simply transferred into black plates for reading in the fluorometer.

Even more preferable to end-point detection, molecular tags of the present invention may be detected by real-time PCR detection. This homogenous PCR method advantageously reduces analysis time, manipulations and contamination risks compared to end-point detection.

Internal Conformation of the Nucleic Acid Tags

Tags according to a preferred embodiment of the first aspect of the present invention are composed of a piece of single stranded DNA, the length of which preferably does not exceed 100 nucleotides for foodstuff applications. It may exceed 100 nucleotides for other applications in which human or animal health is not considered at risk (e.g. for non-edible products such as paint, petrochemical products and the like). The nucleic acid tags are naturally folded into the form of a hairpin by virtue of two short complementary nucleotide sequences added at the 5′ and 3′ ends of the molecule. Among other advantages, this conformation is useful in stabilizing the molecule and protecting it from degradation, thus ensuring a longer half-life as is further illustrated in Example 13 below.

These complementary nucleotides anneal together to form the stem structure of the hairpin, the loop being formed by the intervening nucleic acid sequence. The length and nucleotide composition of the stem is chosen so that the tag will adopt its intended hairpin structure under normal conditions of use (temperature, salt concentration of the tagged product, etc.). Proper folding can be determined theoretically with the use of suitable nucleic acid folding software such as mFOLD software written by Dr. M. Zuker, Renssaeler Institute, available at http://bioinfo.math.rpi.edu/˜mfold/dna/form1.cgi. However, the melting temperature of the stem should be such that the stem will melt at a temperature slightly lower than the annealing temperature of the primers used for amplification (preferably by PCR).

A careful optimisation of nucleotide sequences and preferably a use of non-complementary nucleotides in the loop will help avoid secondary structure formation therein and thereby, as indicated earlier, increase the detection sensitivity with molecular beacons or other probes. One skilled in the art will be able to determine the optimal length of the tag, and/or the sequence of the loop portion and/or the optimal number of non-complementary nucleotides. This optimisation can advantageously reduce and preferably avoid the formation of secondary structure under the normal conditions of use of the tag.

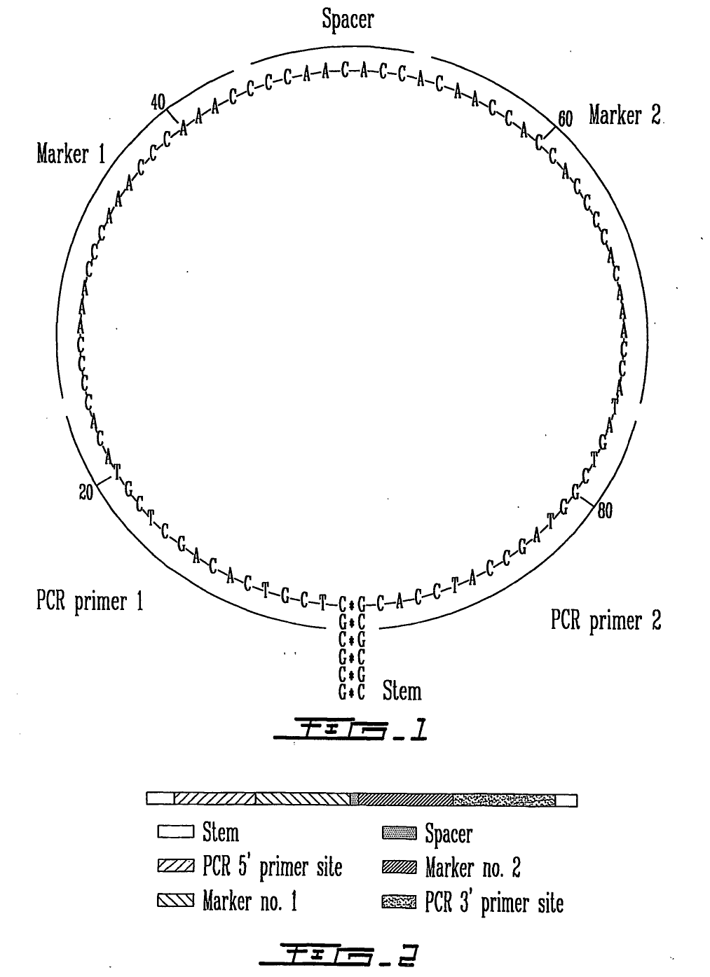

As is illustrated in FIG. 1, the sequences forming a stem according to a preferred embodiment are made of between about 4 to about 8 nucleotides (6 nucleotides are illustrated in FIG. 1), lengths shown to promote hairpin structure of the tag under normal conditions of use. The stem appearing in FIG. 1 are mostly comprised of C and G. As known, C and G form 3 hydrogen bonds and their interaction is therefore more stable than an A-T interaction (forming 2 hydrogen bonds). This conformation of the stem proved effective in achieving the desired hairpin shape of the nucleic acid tag. Other conformations comprising non-complementary sequences and at least one pair of complementary sequences might also be suitable for applications disclosed herein.

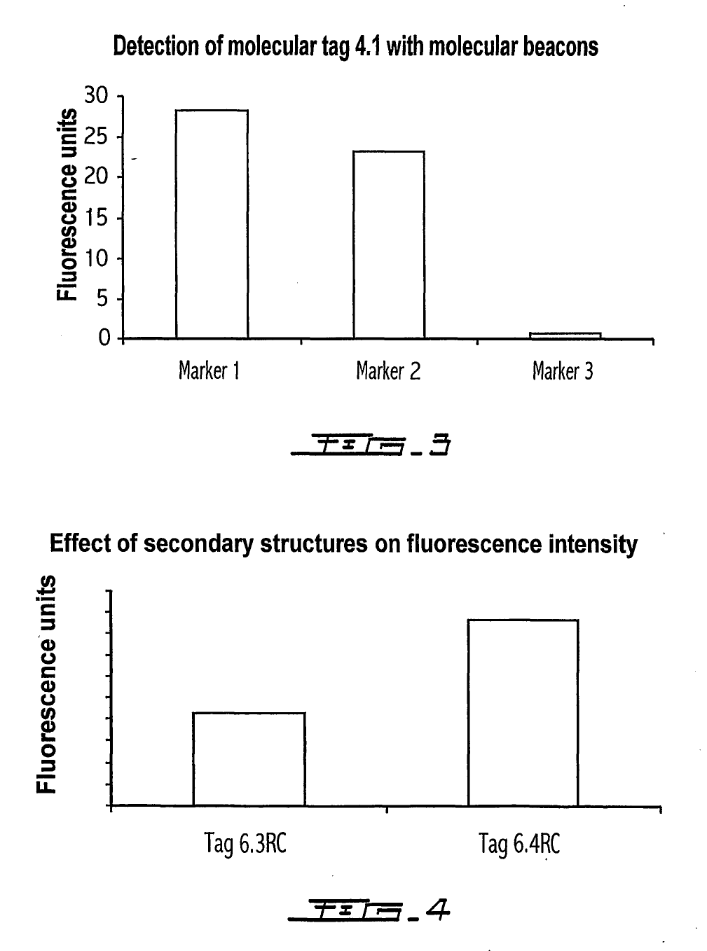

As is illustrated in FIG. 2, internal to the stem sequences are two sequences that are complementary to PCR primers to allow PCR amplification of the nucleic acid tags. When molecular beacons are used in conjunction with PCR amplification, the length of the PCR product is preferably less than about 300 base pairs and most preferably about 80 to 150 base pairs. Although the present embodiment is exemplified with PCR primers, other amplifications means could also be used to amplify molecular tags of the present invention. In addition, if molecular beacons are not used, the length of the amplification primer target sequence could be changed in accordance with the specific needs in accordance with the known laws of thermodynamics and the like.

Internal to these PCR primers binding sites is at least one marker sequence, each being 18 to 25 nucleotides long. Marker sequences having a length comprised between 18 and 25 nucleotides are preferred when molecular beacons (U.S. Pat. No. 5,925,517) are used to identify the markers: 18 and 25 nucleotides is the recommended length of molecular beacon probe sequence (Tyagi et al., 1996, “Molecular beacons: probes that fluoresce upon hybridization” 14 Nature Biotechnol. 303-308). Shorter fragments would result in less fluorescent signal, whereas longer fragments would not increase significantly the signal. In the embodiment shown in FIG. 2, a spacer separates the two markers, and this marker is as long as the size limitation for the whole nucleic acid tag Will allow (generally 100 nucleotides). When molecular beacons are used to determine the sequence of the amplification products, the length of the spacer should be adjusted to make hybridization with two molecular beacons easier (i.e. sufficient space should be provided between the quencher of the first beacon and the fluorophore of the second beacon. Again, because the total length of the nucleic acid tag preferably does not exceed 100 nucleotides for certain applications, while the length of the spacer should be as long as possible, it is nevertheless limited. The presence of a spacer between the two molecular binding sites in the exemplified tag is not necessary for all types of nucleic acid tags. Thus, a person of ordinary skill will be able to adapt the design thereof to their particular needs. For example, the spacer region could be used as yet a further marker sequence or PCR primer binding sequence.

To avoid undesirable internal folding of the nucleic acid tags, the markers and spacer are preferably made of only 2 non-complementary bases (such as cytosine and adenosine, or cytosine and thymine). However, the nucleic acid tags can also be composed of all four nucleotides, and the sequence thereof adjusted according to conventional means to limit or avoid secondary structure formation (e.g. software programs which predict the potential for secondary structure formation and the melting temperature associated with same) and enable satisfying PCR product detection. Nevertheless, in a particularly preferred embodiment, the markers are composed exclusively of non-complementary bases. This design enables a particularly efficient detection with molecular beacons as compared to markers comprising complementary bases, possibly due to the formation of strong internal secondary structures. Of course, the person of ordinary skill could design a tag having sequences of all four nucleotides, but chosen as to avoid the formation of strong secondary structure.

There are 4 possible combinations of two non-complementary bases, thus, when the markers have a length of 25 nucleotides, 134×10⁶ different markers (4×2²⁵=134×10⁶). Consequently, according to the presently exemplified nucleic acid, which bears two markers, the total number of possible nucleic acid tags reaches 1.8×10¹⁶. Hence, the number of different nucleic acid tags provided with this preferred embodiment of the first aspect of the present invention is for practical purposes indefinite.

Consequently, while the nucleic acid tag is relatively small and simple, it nevertheless provides an impressive complexity.

While the markers bore by the nucleic acid tags are preferably unique, the PCR primer binding sites may be shared by a large number of nucleic acid tags, allowing universal amplification. It would be, for example, possible to allocate a certain set of PCR primers for each user of the technology. This would ensure the simple identification of products by legitimate users of the technology, while maintaining the privacy of PCR [symbol] key [symbol] towards other users.

The presence of two markers on the same nucleic acid tags allows for a double identification. For example, one marker could identify a certain company, and the second marker could identify one of its products. Alternatively, the two markers could identify a certain product and a particular lot of this product, or a product and production date. The possibilities are numerous and are adaptable by a person of ordinary skill to meet particular needs.

Tagging of Products

After synthesis using usual DNA synthesizing technology, the nucleic acid tags are resuspended in a suitable buffer (for instance, water, 10 mM Tris or TE) at an adequate concentration (more or less 500 μM). The buffer used is not critical in terms of long term storage of the nucleic acid tag stock solution. Any buffer that is suitable for DNA conservation is adequate. The only limitation is that the buffer used must be non-toxic when the nucleic acid tags will be used to tag food products. It will be understood that for certain applications, an inhibitor of nucleases might be added to the buffer solution. The concentration of the stock solution is not critical either. It should be adjusted so that the solution is easy to manipulate. This will ultimately depend on the volume of product to be tagged. Dilutions of the stock solution can be made to allow easier handling.

To tag liquid products (fruit juices, milk, gasoline and the likes), the nucleic acid tag solution can be added directly to the product and mixed thoroughly.

To tag fruit juices, the nucleic acid tags are preferably added at a final concentration of 10⁵ molecular tags per microliter of juice (3.5 pg of tag/ml orange juice). This amount of nucleic acid tags correspond to 166 fmol (166×10⁻¹⁵ moles) per liter, or 0.005 part per trillion.

To tag gasoline, a small volume of suitable buffer, such as a common gasoline additive, containing the nucleic acid tags may be added to the gasoline and mixed. These additives include: antioxidants, anti-corrosion agents, chelating agents, anti-emulsifiers, anti-knocking and octane booster agents, colors, drag reducers, etc.

Of course, a person of ordinary skill will be able to select a suitable buffer for the product to be tagged in order to meet the desired needs.

Extraction and Purification of Tags

The nucleic acid tags are extracted from the tagged product. A purification/extraction step is preferably added to detect tags in certain products: inhibition tests have shown that orange juice for instance has a strong inhibiting activity on PCR. Because of this, the nucleic acid tag concentration used is too low to be detected by adding a small amount of juice directly to the PCR reaction, and purification is advisable. Other tagged products such as paper, may not require such a purification step.

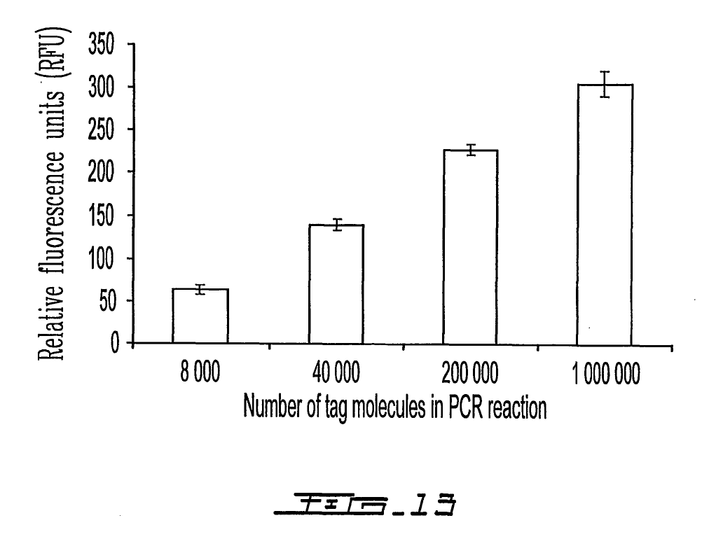

Any suitable nucleic acid purification/extraction method may be used. Advantageously, extraction methods that are simple and easy to automate, can be used effectively in accordance with the present invention. In particular, the biotin/streptavidin extraction method is appropriate. This method is illustrated in FIG. 12. In panel A, a biotin molecule is covalently attached to the 5′ end of a ssDNA molecular tag of the present invention. When the tagged product is contacted with streptavidin-coated magnetic microparticles, because of the affinity between biotin molecules and streptavidin, the biotin-labeled tags bind to the streptavidin-coated magnetic microparticles (FIG. 12, panel B). The biotin-labeled tags/streptavidin coated beads complexes are then recovered by means of a rare-earth magnet. This method works in a variety of products and the presence of biotin may prevent the risks of recombination of the tags into genome of living organisms.

Other examples of appropriate extraction methods include using commercial kits such as the Qiagen Inc. QIAquick™ Nucleotide Removal kit. This kit may be used for the purification/extraction of nucleic acid tags from liquid products such as fruit juices, milk products and gasoline. For this purpose, a volume of 100 μl of the tagged product is obtained and extracted following instructions provided with the kit. Final elution is performed in 100 μl of suitable elution buffer (usually distilled water or a 10 mM solution of Tris-hydroxymethyl amino-methane buffered at pH 8.0 with hydrochloric acid). When the tagged product contains large-sized particles, such as pulp in orange juice, these particles may be removed from the sample by centrifugation either before extraction of immediately after the addition of buffer PN (see kit's instructions). At the end of the extraction procedure, 1 μl of the eluted DNA solution is usually sufficient to obtain a PCR product easily detectable by any conventional PCR product detection method.

PCR Amplification of the Nucleic Acid Tags

After purification, 1 μl of the eluent (the same amount can be used in cases where no purification is effected) is amplified by PCR using a standard protocol and the appropriate PCR primers. The PCR primer binding sites being preferably universal, the same primers are suitable to amplify all the nucleic acid tags used by a certain user.

To increase the sensitivity of the PCR amplification when very low concentrations of nucleic acid tags are used, asymmetric PCR may be preferred. The best sensitivity was obtained with asymmetric PCR combined with a 50-cycle program. Using this setup, the fluorescent signal was boosted up to 10 times. However, this increase is likely to be accompanied by a reduction in the perceived accuracy of the test. Unspecific PCR products are also formed during such long programs, and these might interfere with the sensitive detection required. For these reasons, it might be preferable to use a PCR program of 40 cycles or less.

In asymmetric PCR, the concentration of one primer is reduced while the concentration of the other is increased. This results in the preferential accumulation of a single stranded product. Since the molecular beacons used for the detection of the products react more strongly with single stranded DNA, the sensitivity of the assay is increased. This approach allows the use of lower concentrations of nucleic acid tags in the tagged product, leading to less potential for toxicological problems and increased difficulty of detection by unauthorized users.

The present invention is illustrated in further details by the following non-limiting examples:

### EXAMPLE 1

### Conventional Amplification Procedure

The following PCR mix was used:

Final concentration

| | | |
|---|---|---|
| 10× PCR buffer from Qiagen inc. | 1× | |
| 10 mM dNTPs ™ (Roche inc.) | 0.2 | mM |
| 25 μM Forward primer | 1.0 | μM |
| 25 μM Reverse primer | 1.0 | μM |
| 5 U/μl HotStart Taq polymerase ™ (Qiagen) | 1 | U |
| DNA sample | 1 | μl |

The PCR mix was subjected to temperature cycling in a Perkin-Elmer™ 9700 thermocycler using the following program:

Denaturation and activation of HotStart Taq™ 95° C., 15 min

40 cycles of the following:

| | |
|---|---|
| Denaturation | 94° C., 10 sec |
| Annealing | 55° C., 15 sec |
| Extension | 72° C., 5 sec |

After amplification, 5 μl of amplified products were loaded onto a 15% polyacrylamide gel and run at 200V, 60-min. The gel was stained in ethidium bromide and photographed.

### EXAMPLE 2

### End-Point Amplification wherein Molecular Beacons are Added After Amplification

The following molecular tag was used:

(SEQ ID NO:1)

GCGCGCTCGTCACAGCTCGTACACCCCAAACCCAAACCCAAACCCCAAC
ACCACAACCACCACCCCACAAACCATAGTCGGTAGCCATCCAC GCGCGC.

It was asymmetrically amplified using the following PCR Mix:

| | | |
|---|---|---|
| 10× PCR Buffer from Qiagen | 7.0 | μl [1×] |
| 10 mM dNTPs ™ from | 1.4 | μl [200 μM] |
| Roche Diagnostics | | |
| 25 μM 5′-primer from | 0.28 | μl [0.1 μM] |
| Life Technologies | | |
| (TCGTCACAGCTCGTACAC; SEQ | | |
| ID NO: 2) | | |
| 25 μM 3′-primer from | 2.8 | μl [1 μM] |
| Life Technologies | | |
| (GTGGATGGCTACCGACTA; SEQ | | |
| ID NO: 3) | | |
| 5U/μl HotStart Taq ™ | 0.5 | μl [10 units] |
| from Qiagen | | |
| Ultrapure ™ sterile water | 58.02 | μl |
| 2.5 × 10⁶ molecular tags | 2.5 | μl |
| having SEQ ID NO: 1. | | |

The amplification was carried out in a Perkin-Elmer 9700™ thermocycler as described in Example 1.

### EXAMPLE 3

### End-Point Detection wherein Molecular Beacons are Added After Amplification

The amplification reaction mixture obtained in Example 2 was divided into 3 volumes of 20 μl. Three reading buffers were prepared, each containing a different molecular beacon. A first molecular beacon for marker 1, the sequence of which appears in FIG. 1 (fluoroscein (hereinafter “FAM”)—ccgag CCCAAACCCAAACCCAAACCC ctcgg—DABCYL; SEQ ID NO: 4), a second molecular beacon for marker 2, the sequence of which appears in FIG. 1 (FAM—cgcac CMCCACCACCCCACAAACCA gtgcg—DABCYL; SEQ ID NO: 5) and a molecular beacon for a third marker not present in molecular tags having SEQ ID NO: 1 (TAMRA—caggc CAACCACACCACACAACACCA gcctg—DABCYL; SEQ ID NO: 6).

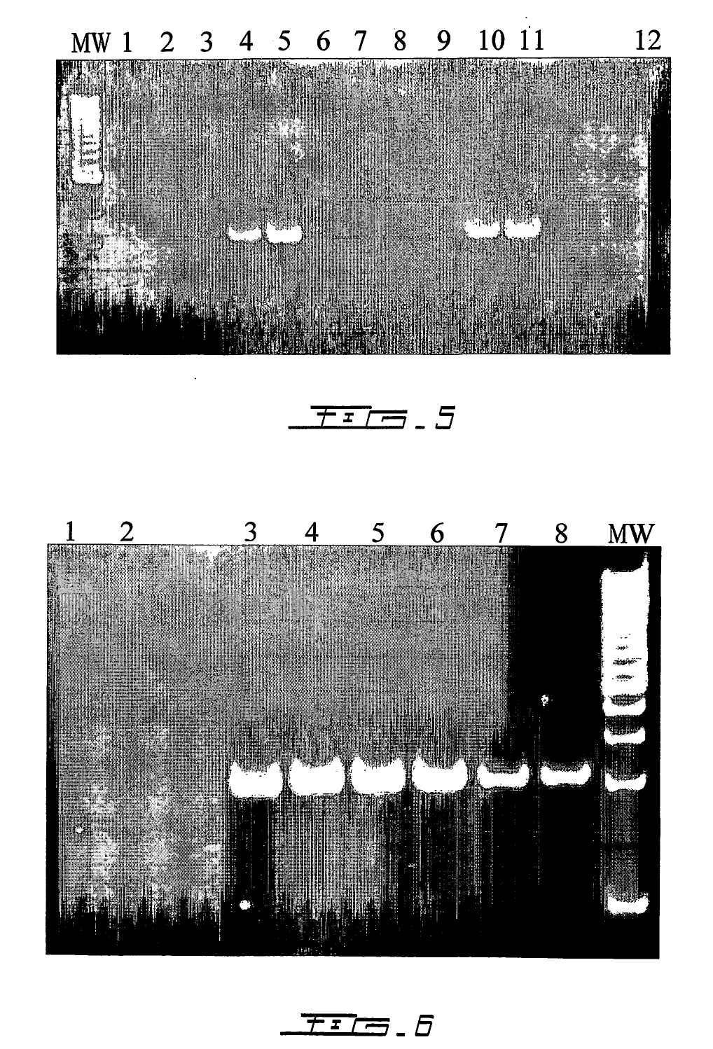

To each sample of amplification reaction, 30 μl of one of the three specific reading buffer was added (100 mM Tris-HCl, pH 8.0; 5.5 mM MgCl2; 0.3 μM molecular beacon). All samples were submitted to a denaturation/annealing cycle (95° C.-2 min; 72° C.-10 sec; 0.1° C./sec to 45° C.; 25° C. forever). The totality (50 μl) of each sample was then transferred in a black Costar™ 96-well plate (Corning). The fluorescence was read at room temperature at the excitation wavelength of 485 nm and emission wavelength of 535 nm. The fluorescence results are presented in FIG. 3.

### EXAMPLE 4

### Amplification Procedure for Real-Time Detection

The PCR reactions were set up as indicated in Table 1, using appropriate combinations of molecular tags, PCR primers and molecular beacons in a final volume of 25 μL.

TABLE 1
PCR reaction mixture

| REAGENT | FINAL CONCENTRATION |
|---|---|
| Qiagen PCR buffer* | 1× |
| dNTPs | 0.2 mM |
| PCR primer (forward) | 0.6 μM |
| PCR primer (reverse) | 0.6 μM |
| Molecular beacon | 0.3 μM |
| MgCl₂ | 2.5 mM* |
| Qiagen HotStarTaq ™ DMA polymerase | 1 U |
| Molecular tag | 10⁸, 10⁵ or 0 molecules / |
| | reaction |

*1.5 mM MgCl₂ was already present in 1× PCR buffer; addition of 2.5 mM MgCl₂ brought the final concentration of this reagent to 4 mM.

The PCR reaction was run in a Biorad iCycler iQ™ real-time PCR unit with the following parameters: initial denaturation at 95° C., 10 min; and 40 cycles of 94° C., 30 sec, 55° C., 30 sec, 72° C., 30 sec. All PCR reactions were done in duplicate.

### EXAMPLE 5

### End-Point Amplification and Detection where Molecular Beacons were Added to PCR Tubes Prior to Amplification

The PCR reactions were set up as indicated in Table 1, using appropriate combinations of molecular tags 9.1 (5′ GGGCCCAGGTCTCTGCCAAGTGTTTAGCCTGGAGGAAGGTGGGGATGACG TCATGGACTGAGCGAMCTTATCGGAACGGGCCC; SEQ ID NO. 9), PCR primers (forward primer: 5′AGGTCTCTGCCMGTGTTT; SEQ ID NO. 10; Reverse premier: 5′GTTCCGATMGTTTCGCTC; SEQ ID NO. 11), and and molecular beacons (FAM—cctcga gaggaaggtggggatgacgtca tcgagg—DABCYL; SEQ ID NO. 12) in a final volume of 25 μL.

TABLE 2
PCR reaction mixture

| REAGENT | FINAL CONCENTRATION |
|---|---|
| Qiagen PCR buffer | 1× |
| dNTPs | 0.2 mM |
| PCR primer (forward) | 1.0 μM |
| PCR primer (reverse) | 1.0 μM |
| Molecular beacon | 0.3 μM |
| MgCl₂ | 2.5 mM* |
| Qiagen HotStarTaq ™DNA polymerase | 1 U |
| Molecular tag 9.1 | 800; 40,000; 200,000; or 10⁸ / |
| | reaction |

*1.5 mM MgCl₂ was already present in 1× PCR buffer; addition of 2.5 mM MgCl₂ brought the final concentration of this reagent to 4 mM.

The PCR reaction was run in a thermal cycler Perkin Elmer™ 9700 with the following parameters: Initial denaturation at 95° C., 15 minutes; and 40 cycles of 94° C., 30 sec, 55° C., 30 sec. 72° C., 30 sec. The program was ended by a final extension step at 72° C., 5 minutes.

This program was followed by 1 cycle of the following sequence: 94° C., 30 sec, 58° C., 30 sec and 25° C., infinite duration (i.e. until actual transfer to black plate for fluorescence reading).

Ten microliters of each reaction were then transferred to a black Costar™ 384-well plate and read in a fluorometer Gemini™ XS.

FIG. 13 illustrates the fluorescence read as a function of the number of molecular tags that were initially in the PCR reaction.

### EXAMPLE 6

### Real-Time Simple and Multiplex PCR Detection Procedures

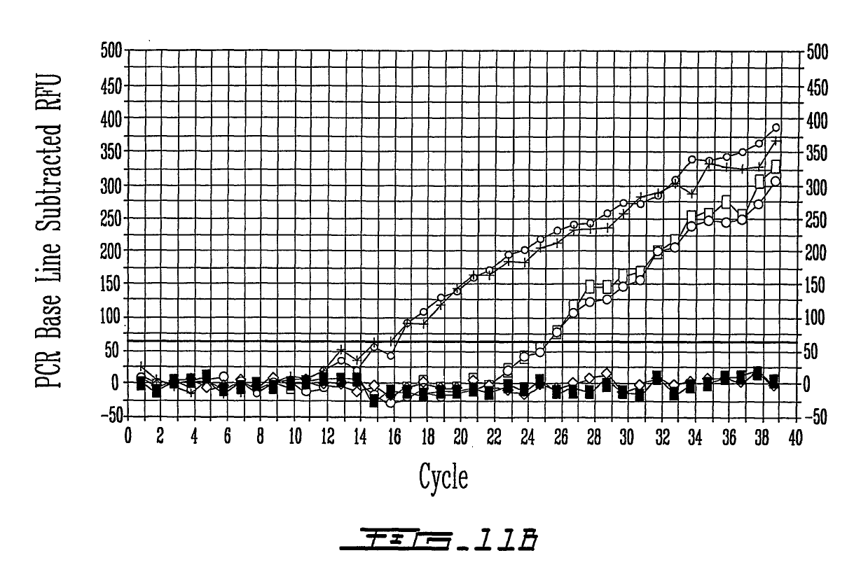

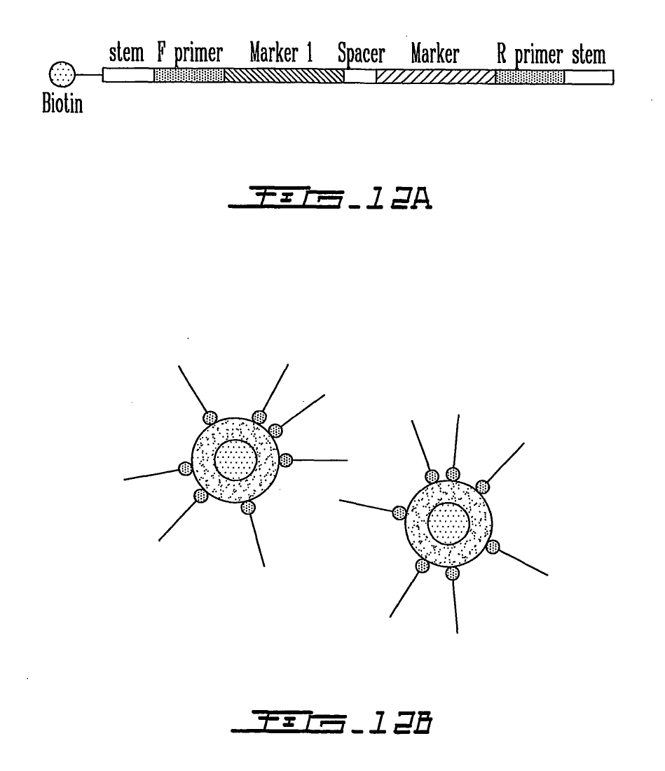

For the real-time detection, two tags were used: molecular tag 11.1 (5′cgcgcATTCAGTCCATGGCAGGTtcgtacaccactcaagcctcgcttagctcAGAMTAAC CGGACACGCgcgcg; SEQ ID NO. 13; Forward primer: ATTCAGTCCATGGCAGGT: SEQ ID NO. 14; Reverse primer: GCGTGTCCGGTTATTTCT: SEQ ID NO. 14) was detected with a molecular beacon labeled with FAM (FAM—ccggg accactcaagcctcgct cccgg—DABCYL: SEQ ID NO. 14), and molecular tag 9.8 was detected with a molecular beacon labeled with Texas Red. Each molecular tag was first amplified and detected individually (FIG. 9 and FIG. 10). The two tags were then combined together and detected in a single multiplex reaction (FIG. 11). In each experiment, two tagged samples containing different quantifies of molecular tags were used, namely 10⁸ and 10⁵ molecules. Negative PCR controls (0 molecule per PCR reaction) were also included in every experiment.

FIG. 9 presents results of real-time PCR detection of molecular tags 11.1 with FAM-labeled molecular beacons. In this figure, the blue/purple traces show the progressive amplification of the targeted sequences of the molecular tags in the duplicate samples that initially contained 10⁸ molecular tags. Similarly, the red/yellow traces show the progressive amplification of the targeted sequences of the molecular tags in the duplicate samples that initially contained 10⁵ molecular tags. The dark green/light green traces represent that of the duplicate samples that initially contained only the negative PCR control. FIG. 10 presents results of real-time PCR detection of molecular tag 9.8 with Texas-Red-labeled molecular beacons. In this figure, the red/yellow traces show the progressive amplification of the targeted sequences of the molecular tags in the duplicate samples that initially contained 10⁸ tags molecules, the dark green/light green, that of the duplicate samples that initially contained 10⁵ molecular tags/PCR reaction, and the blue/turquoise traces, that of the duplicate samples that initially contained only the negative control.

FIG. 11 presents results of a multiplex detection of molecular tag 11.1 and molecular tag 9.8. Both molecular tags were added and detected simultaneously in PCR reactions containing both sets of PCR primers and both molecular beacons, namely MB 11.1 labeled with FAM; and MB 9.8 labeled with Texas Red. Panel A presents detection results of molecular tag 11.1 and panel B presents detection results of molecular tag 9.8. In these panels, the pink/red traces show the progressive amplification of the targeted sequences of the molecular tags in the sample that initially contained 10⁸ molecules of each tag. The blue/purple traces show the progressive amplification of the targeted sequences of the molecular tags in the sample that initially contained 10⁵ molecules of each molecular tag, and the red/yellow traces that of the sample that initially contained only the negative control.

### EXAMPLE 7

### Use of Molecular Tags to Quantify Proportion of Specific Component in a Mixture

Two volumes of nanopure™ water of 4.5 mL of were prepared: one was tagged with 10⁸ molecular tag/μL whereas the other was not. Aliquots of the two volumes were then combined in various proportions to form final samples of 1.5 mL. The tags were then extracted and the amount present in each sample was calculated by real-time PCR. These values were then used to trace back the proportion of the tagged water used to form each final sample. For these experiments, the PCR reagents and cycle parameters were as described in Example 4. All PCR reactions were done in duplicate. This experiment demonstrated the usefulness of molecular tags to trace back approximately which quantity of a specific component went into the manufacture of a final mixture. The differences between the theoretical values and the calculated values obtained with this test and presented in Table 3 are well into error margins obtained with comparable real-time quantitative PCR tests (e.g. tests used to determine the amount of Hepatitis B virus in blood serum wherein the theoretical values ranged from 31% to 111% of the calculated values (Brechtbuehl, 2001)).

TABLE 3
Summary of quantification data

| | Theoretical amounts | | Calculated amounts | |
|---|---|---|---|---|
| | | Amount of | | Amount of |
| | Proportion | tag in | Proportion | tag in |
| | of tagged | sample | of tagged | sample |
| Sample # | water (%) | (molecules/μL) | water (%) | (molecules/μL) |
| 1 | 0.00 | 0 | 0.00 | 0 |
| 2 | 0.01 | 5.0 × 10³ | 0.02 | 9.1 × 10³ |
| 3 | 0.10 | 5.0 × 10⁴ | 0.14 | 7.1 × 10⁴ |
| 4 | 0.50 | 2.5 × 10⁵ | 0.62 | 3.1 × 10⁵ |
| 5 | 1.0 | 5.0 × 10⁵ | 1.4 | 7.1 × 10⁵ |
| 6 | 10 | 5.0 × 10⁶ | 14 | 6.9 × 10⁶ |
| 7 | 50 | 1.3 × 10⁷ | 36 | 1.8 × 10⁷ |
| 8 | 100 | 5.0 × 10⁷ | 128 | 6.4 × 10⁷ |

### EXAMPLE 8

### Tagging of Apple Juice with Biotin-Labeled Molecular Tags

Biotin-labeled molecular tags were spiked into Oasis™ apple juice at a final concentration of 10⁶ molecular tags per ml of juice. The biotin-labeled molecular tags were then extracted as follows.

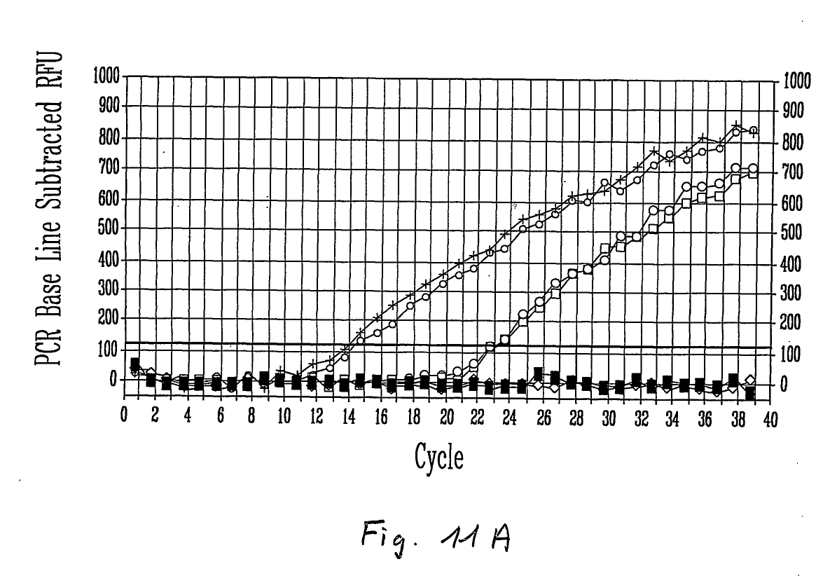

100 μL of buffer A were added to an equal volume of tagged apple juice. One microliter of a 1% stock solution of High binding Streptavidin-coated magnetic microparticles™ (Seradyn inc., USA) was added to the mixture in a 0.2 ml sterile PCR tube and left at room temperature for 1 hr in the dark. The tubes were then placed over a rare earth magnet for 10 sec to allow the beads to form a tight pellet at the bottom of the tube. The supernatant was removed followed by addition of 200 μl of buffer A and vortexing. The slurry was transferred to a fresh PCR tube. The washing procedure was repeated 4 times. After final wash, the pellet was resuspended in 5-10 μl of distilled H2O and stored at −20° C. until used. The stored bead pellets were then thawed and amplified according to the PCR method described in Example 2. Amplified molecular tags appear in FIG. 8 wherein lane MW contains molecular weight markers, lanes 1-2 are empty, lanes 3-4 contain extraction products without microbeads and lanes 5-6 contain amplification products with microbeads.

### EXAMPLE 9

### Tagging of Unleaded Gasoline

Molecular tags diluted in 100 μl of water were added to 10 ml of unleaded gasoline at a final concentration of 10¹¹ molecular tag/μl. The tagged gasoline sample was vortexed thoroughly and kept in a sealed brown glass container at room temperature.

Extractions were performed with the Qiagen Nucleotides Removal Kit™ by mixing 100 μl of gasoline with 10 volumes of buffer PN. The suggested protocol of the manufacturer was followed thereafter. Elution was performed with 100 μl of EB buffer and the extracted DNA was kept at −20° C. until needed in 1.5 mL eppendorf™ tubes.

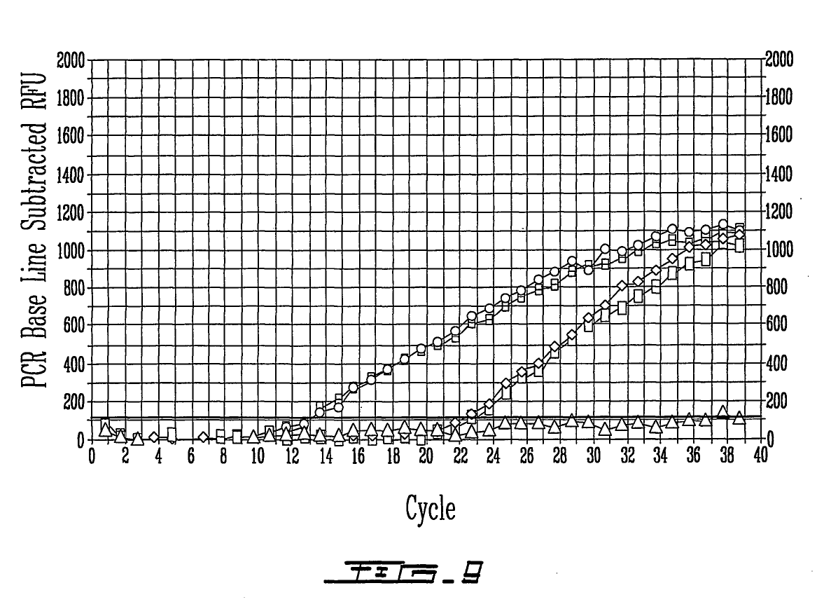

The stored extracted DNA was then amplified according to the PCR method described in Example 2. Amplified molecular tags appear in FIG. 6 wherein lane MW contains molecular weight markers; lanes 1 and 2 contain negative controls comprising buffer only, and gasoline and buffer, respectively; lanes 3 to 8 contain amplified tags that have remained in gasoline one, 2, 4, 8, 11 and 15 weeks, respectively.

### EXAMPLE 10

### Tagging of Ground Beef

Lean ground beef was purchased at a local grocery store and divided into 100 g samples in Zip-Loc™ bags. Each sample was formed into a ball and flattened. Some samples were then mixed with the desired amount of molecular tag, namely 10¹¹ molecular tags per 100 g of beef, diluted in 1 ml sterile H₂O. This was done by dispersing 500 μl of the solution on each side of the meat patty with a 2.0 ml sterile plastic pipet. Some samples were untagged and kept as controls. The meat was then mixed thoroughly and the patty was reformed. Small samples (0.5 g) were taken from the center of the patty with a sterile micropipet tip and kept in 1.5 ml eppendorf tubes. A portion of the tagged ground beef was then frozen at −20° C. for 3 days.

The meat was thawed one patty at a time by microwaving 5-7 min at medium power and then cooked one at a time at medium heat 5 min on each side with 1 tbsp. of canola oil. Untagged control samples were cooked first.

The tagged ground beef samples previously frozen or not were then subjected to extraction. The molecular tags were extracted with The Qiagen Nucleotides Removal Kit™ according to the manufacturer's instruction except for minor modifications in the sample preparation. To 0.5 g samples in 1.5 ml tubes, 700 μl of PN buffer were added followed by centrifugation at 13,000 rpm, 1 min. The meat was removed with a toothpick and the sample was centrifuged again at 13,000 rpm, 1, min. The manufacturer's instructions were followed thereafter. Final elution was performed with 70 μl of buffer EB and the purified DNA was stored at −20° C.

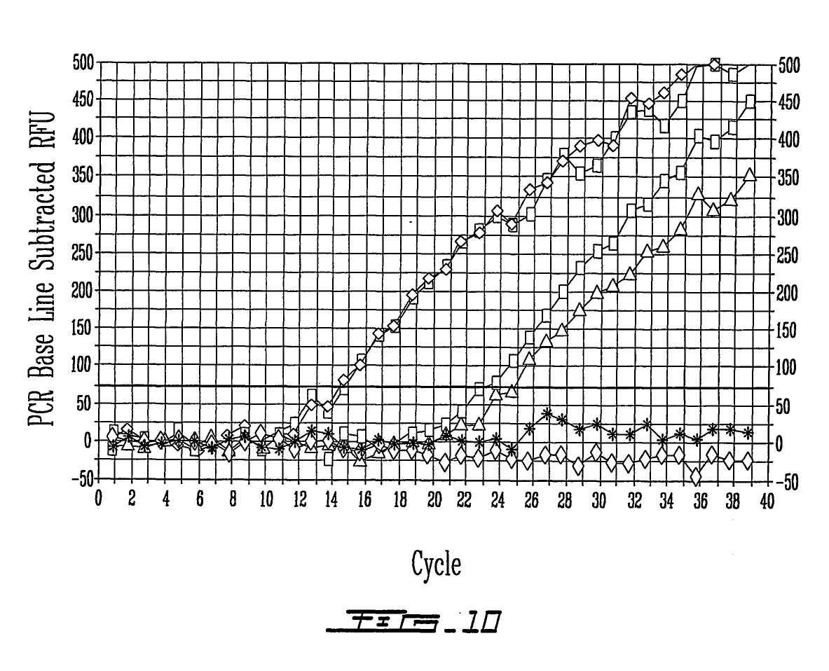

The extracted molecular tags were then amplified by PCR according to Example 2 prior or after cooking. Photographs of the amplified products are shown in FIG. 7 wherein the lanes contains the following: lane MW molecular weight markers; lane 1 negative control for extraction; lane 2 the negative control for PCR; lane 3 amplification products from raw untagged meat; lane 4 amplification products from cooked untagged meat; lane 5 amplification products from tagged uncooked meet, lane 6 amplification products from tagged cooked meet; lanes 7-8 amplification products from frozen untagged raw meat; lane 9 amplification products from frozen untagged cooked meat; lane 10 amplification products of frozen tagged uncooked meet; lane 11 amplification products from tagged frozen cooked meet; and lane 12 positive control for PCR (10⁵ molecular tags).

### EXAMPLE 11

### Effect of Secondary Structures on Fluorescence Intensity

The effect of the secondary structures of the molecular tags on the fluorescence observed with molecular beacons was investigated by using the following molecular tags:

(SEQ ID NO:7)

6.3RC
5′CATTCCTGACCGTTACGACATTCGTTCACATTAGTTATCGCATTTCG
GGAGCTAATGAACCTGCGGCACGT;
and

(SEQ ID NO:8)

6.4RC
5′GCTTACAGCATTGCCAGTCATTTGTTCACATTAGTTATCGCATTTCGTC
GACGGGGTCCAAGTAATCGAGG;

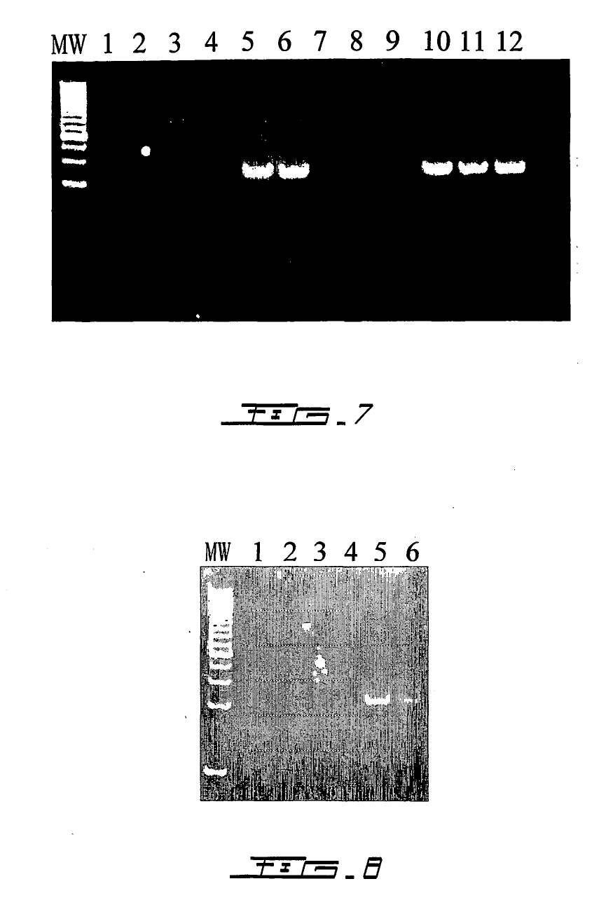

The results of this experiment appear in FIG. 4. The core of molecular tags 6.3RC and 6.4RC contain an identical sequence recognized by molecular beacon Pth6 (FAM—gcagag AATGCGATAACTMTGTGAA ctctgc—DABCYL; SEQ ID NO: 8). The regions flanking the molecular beacon binding sites were chosen randomly for tag 6.3RC but were carefully optimized in tag 6.4RC to avoid the formation of secondary structures.

For each tag, 0.6 μM were mixed with 0.3 μM of molecular beacon in a buffer containing 10 mM Tris, pH 8.0 and 4 mM MgCl2. Fifty microliters of each solution were transferred into wells of a black Costar™ 96-well plate (Corning inc., USA) and the fluorescence was read in a Molecular Devices Gemini XS™ fluorometer using the following parameters: 485 nm excitation wavelength, 535 nm emission wavelength, 515 nm cutoff wavelength.

### EXAMPLE 12

### Effect of Additional Nucleotides on Molecular Tags in Regions Outside of PCR Primers on Amplification Efficiency

Molecular tags with 6 additional nucleotides (lanes 1-5) or with only 3 additional nucleotides (6-10) were amplified by PCR starting with increasing numbers of molecules in each PCR reaction. Results are presented in FIG. 5. Lane MW: molecular weight markers; lanes 1 and 6: 1 tag molecule; lanes 2 and 7: 10 tag molecules; lanes 3 and 8: 10² tag molecules; lanes 4 and 9: 10³ tag molecules; lanes 5 and 10: 10⁴ tag molecules; lane 11: positive PCR control (10⁵ tag molecule with 6 additional nucleotides outside PCR primers); lane 12: negative PCR control.

The application efficiency was similarly tested with tags that did not possess additional nucleotides outside the PCR primer binding sites (results not shown). These results showed that the addition of additional nucleotides outside of PCR primer binding sites in molecular tags significantly increased their amplification efficiency.

### EXAMPLE 13

### Molecular Tags Half-Life

Experiments have shown that the molecular tags of the present invention having a hairpin shape can survive in orange juice (Oasis™ pure premium with pulp) and pineapple juice (Del Monte™) for a period of up to 8 weeks without any detectable degradation as monitored by PCR. These experiments have not been prolonged any further due to the expiration date of the tagged products being reached.

In gasoline, these nucleic acid tags have survived up to 6 weeks without apparent degradation as is illustrated in FIG. 6. At various time intervals, samples of tagged gasoline were taken, extracted and amplified by PCR according to the procedure described in Example 2. Lane 1: negative control for extraction; lane 2: untagged gasoline; lane 3: 1 week; lane 4: 2 weeks; lane 5: 4 weeks; lane 6: 8 weeks; lane 7: 11 weeks; lane 8: 15 weeks; lane MW: molecular weight markers. Similar results were obtained in distilled water.

It has been shown that nucleic acid tags of the present invention that did not have a hairpin shape degrade in distilled water. Degradation appeared to increase steadily over a period of 8 weeks.

No degradation was observed for linear nucleic acid tags spiked into the orange juice tested.

This shows that depending on the product to be tagged, it might be desirable to protect the molecular tag from degradation. The hairpin structure provided by the exemplified molecular tag which comprises a double-stranded region protecting the ends of the tag constitutes one non-limiting example of such protection against degradation. Of course, a person of ordinary skill will understand that other means of protecting of the tag from degradation are possible. Non-limiting examples of such tag protections include extra sequences at the ends of the tag that can act similarly to telomeres or single stranded oligos which bind to the ends.

The following examples relate to new uses for molecular tags.

### EXAMPLE 14

### Identification of Raw Product Suppliers

Raw products used in the manufacture of goods (food stuff and others) often come from various suppliers. It is often desirable to trace from which of these suppliers the raw materials used for the manufacture of a specific (and perhaps defective) product originate. For this purpose, a specific molecular tag can be assigned to every supplier, the tags being then added to the raw materials as they are received, or are added prior to expedition and identified in the finished product. Examples of raw materials that could be tagged include fresh fruits, flour, chemicals, etc.

### EXAMPLE 15

### Tagging of Production Lines

In large-scale production facilities, many production lines are often run in parallel and the finished products are pooled together. It is often desirable to determine from which production line originates a specific (and perhaps defective) product. For this purpose, a molecular tag may be assigned to each production line, and added to the products at a convenient step in the manufacturing process. These tags can then be identified in the finished product. Examples include pasteurizers, incubators, mixers, etc. run in parallel.

### EXAMPLE 16

### Control of Processing Time

Often times, products must spend a defined amount of time at a certain manufacturing step (such as pasteurisation, mixing, etc.). It is often desirable to determine if a finished product has spent the required amount of time at that step. For that purpose, a defined amount of a first molecular tag can be added to the product prior to the beginning of the timed procedure. A second tag is added at a defined rate during the timed procedure. By knowing the amount of the first tag added and the amount and rate of addition of the second tag, one can determine if the proper processing time was followed. For example, the rate of addition of the second tag could be chosen so that, at the end of the processing procedure, the amount of the second tag is equal to that of the first tag if the duration of the procedure is correct. Estimation of the relative proportion of the two tags in the finished product allows one to determine if the duration of the timed procedure was correct.

### EXAMPLE 17

### Quantification of Raw Product

In many circumstances, producers are bound to obtain raw materials through a certain supplier according to the terms of exclusivity contracts. In many cases, the supplier may want to make sure that the producer does not obtain raw materials from other suppliers. For this purpose, a molecular tag may be added to the raw products of that supplier before shipment to the producer. Detection of the tag in the finished product will help to ensure that the raw materials used indeed came from the right supplier. Quantification of the tag will help to determine if the legally obtained raw materials were mixed with similar materials from other sources.

### CONCLUSIONS

The present invention therefore provides a simple and versatile molecular tag that can be easily detected, provides outstanding degeneracy and can be produced at low cost. In accordance to a specific embodiment, the nucleic acid tag is made of synthetic DNA. The ends thereof may be protected from degradation (by intermolecular or intramolecular priming). Most preferably, the nucleic acid tag is a single-stranded molecule with a base-primed 3′ and 5′ end portion, which avoids the drawbacks associated with nucleic acid molecules which can get integrated in a cellular genome.

The invention further provides very versatile methods of using molecular tags (such as the nucleic acid tags defined herein, as well as others in the art described for other more traditional applications) to monitor qualitatively and quantitatively the manufacturing process of goods.

Although the present invention has been described hereinabove by way of preferred embodiments thereof, it can be modified without departing from the spirit and nature of the subject invention as defined in the appended claims.

### REFERENCES

Brechtbuehl, K., Whalley, S. A., Dusheiko, G. M., Saunders, N. A. 2001. A rapid real-time quantitative polymerase chain reaction for hepatitis B virus. Journal of virological methods 93: 105-113

What is claimed is:

1. A nucleic acid tag for monitoring, detecting or tracing substances comprising said tag comprising:
(a) a single-stranded nucleic acid region;
(b) two ends being capable of pairing with a complementary nucleotide sequence; and
(c) at least one marker sequence having a number of non-complementary nucleotides sufficient to minimize or prevent the formation of secondary structure within said marker under conditions of use.

2. The nucleic acid tag of claim 1, wherein said nucleic acid is DNA.

3. The nucleic acid tag of claim 1 or 2, wherein said tag comprises two marker sequences of sufficient length and separated by a spacer sequence so as to be detectable by at least one molecular beacon.

4. The nucleic acid tag of any one of claims 1 to 3, wherein said ends are complementary to each other and form a stem structure.

5. The nucleic acid tag of any one of claims 1 to 4, having a length shorter than about 1000 nucleotides.

6. The nucleic acid tag of any one of claims 1 to 5, having a length shorter than about 100 nucleotides.

7. The nucleic acid tag of any one of claims 1 to 6, further comprising sequences that are complementary to amplification primers and having a sequence which minimizes or prevents the formation of secondary structure therein under conditions of use.

8. The nucleic acid tag of any one of claims 1 to 7, further comprising a spacer sequence having a sufficient number of non-complementary nucleotides to prevent the formation of secondary structure in said single strand region, internal to said two ends under conditions of use.

9. A nucleic acid tag comprising:
(a) a single-stranded nucleic acid sequence portion having a 5′ end portion and a 3′ end portion;
(b) at least two amplification primer binding sequences in said 5′ end and 3′ end portions;
(c) at least two marker sequences having a length of about 18 to about 25 nucleotides internal to said primer binding sequences; and
(d) a spacer between said marker sequences, wherein said spacer has a length which is sufficient to allow a specific binding of molecular beacons to amplification copies of said marker sequences and wherein the nucleic acid sequences of said primer binding sequences, of said marker sequences and of said spacer are chosen so as to minimize or prevent secondary structure formation.

10. The nucleic acid tag of claim 9 wherein said 5′ end and the 3′ end portions are protected from degradation.

11. The nucleic acid tag of claim 9 or 10, wherein said nucleic acid sequence of said primer binding sequences, said marker sequences and said spacer are made of non-complementary nucleotides.

12. The molecular tag of any one of claims 9 to 11, wherein said single stranded nucleic acid comprises 100 nucleotides or less.

13. The molecular tag of any one of claims 9 to 11, wherein said nucleic acid is DNA.

14. The nucleic acid tag recited in claim 13, wherein said amplification primer binding sequences are PCR primer binding sequences enabling PCR amplification procedure, and further comprising a nested PCR primer binding sequence located internally with respect to said PCR primer binding sequences thereby enabling a carrying out of nested asymmetric PCR for increased detection sensitivity.

15. A method of tagging a substance for its identification comprising:
(a) tagging said substance with a molecular tag as in one of the above-mentioned claims;
(b) releasing the tagged substance in the stream of trade or in the environment;
whereby the substance suspected to contain the tag can be identified by subsequent amplification and qualitative and/or quantitative detection of said molecular tag in the substance.

16. The method for detecting a molecular tag according to of any one of claims 1 to 14 in a substance comprising:
(a) taking a sample of said substance suspected to contain the tag;
(b) submitting said substance to an amplification step;
whereby a detection of an amplification product by a detection probe, positively identifies said molecular tag.

17. The method of claim 16, wherein said amplification step is a PCR amplification and said detection probe is a molecular beacon.

18. Use of a molecular tag for characterizing qualitatively and/or quantitatively at least one procedure of a manufacturing process for manufacturing an end product from at least one raw and/or intermediate product.

19. Use of a molecular tag as in claim 18, wherein said at least one procedure is a mixing procedure comprising:
(a) adding a defined quantity of a specific molecular tag in one of the raw and/or intermediate products, prior to mixture with at least one other raw and/or intermediate product, to obtain a tagged product;
(b) mixing said tagged product with said at least one other raw and/or intermediate product to obtain a mixture;
(c) determining the quantity of said molecular tag in said mixture, whereby the quantity of said tagged product in said mixture can be deduced from the quantity of molecular tag contained in said mixture.

20. Use of a molecular tag as in claim 18, wherein said at least one procedure is a timing of a manufacturing step and comprises:
(a) adding a predetermined amount of said molecular tag at a defined rate to said raw and/or intermediate product during the timed manufacturing step;
(b) determining the quantity of said molecular tag in said raw and/or intermediate product after said timed manufacturing step,
whereby the duration of the manufacturing step can be deduced from said quantity of said molecular tag in said raw and/or intermediate product after said timed manufacturing step.

21. Use of a molecular tag as in claim 20, wherein said manufacturing step is selected from the group consisting of pasteurizing, mixing, heating and cooling.

22. The use according to any one of claims 18-21, wherein said molecular tag is a nucleic acid tag as defined in any one of claims 1-14.

23. A method of identifying a defective production line in a manufacturing process which comprises a pooling of manufactured products from at least two production lines to generate a pooled manufactured product comprising:
(a) adding a specific molecular tag to said manufactured product in each production line prior to said pooling;
(b) identifying a defective pooled manufactured product;
(c) identifying said molecular tag in said defective product, whereby the identity of said molecular tag in said defective product leads to the identification of said defective production line.

24. The method of claim 23 wherein said molecular tag is a nucleic acid tag as defined in any one of claims 1-14.


SEQUENCE LISTING (17 sequences)

SEQ ID NO: 1 - DNA, 98 bp, Artificial Sequence
  Oligonucleotide
gcgcgctcgtcacagctcgtacaccccaaacccaaacccaaaccccaacaccacaaccaccaccccacaaaccatagtcggtagccatccacgcgcgc

SEQ ID NO: 2 - DNA, 18 bp, Artificial Sequence
  Oligonucleotide
tcgtcacagctcgtacac

SEQ ID NO: 3 - DNA, 18 bp, Artificial Sequence
  Oligonucleotide
gtggatggctaccgacta

SEQ ID NO: 4 - DNA, 31 bp, Artificial Sequence
  Oligonucleotide
ccgagcccaaacccaaacccaaacccctcgg

SEQ ID NO: 5 - DNA, 31 bp, Artificial Sequence
  Oligonucleotide
cgcaccaaccaccaccccacaaaccagtgcg

SEQ ID NO: 6 - DNA, 31 bp, Artificial Sequence
  Oligonucleotide
caggccaaccacaccacacaacaccagcctg

SEQ ID NO: 7 - DNA, 84 bp, Artificial Sequence
  Oligonucleotide
gggcccaggtctctgccaagtgtttagcctggaggaaggtggggatgacgtcatggactgagcgaaacttatcggaacgggccc

SEQ ID NO: 8 - DNA, 19 bp, Artificial Sequence
  Oligonucleotide
aggtctctgccaagtgttt

SEQ ID NO: 9 - DNA, 19 bp, Artificial Sequence
  Oligonucleotide
gttccgataagtttcgctc

SEQ ID NO: 10 - DNA, 34 bp, Artificial Sequence
  Oligonucleotide
cctcgagaggaaggtggggatgacgtcatcgagg

SEQ ID NO: 11 - DNA, 75 bp, Artificial Sequence
  Oligonucleotide
cgcgcattcagtccatggcaggttcgtacaccactcaagcctcgcttagctcagaaataaccggacacgcgcgcg

SEQ ID NO: 12 - DNA, 18 bp, Artificial Sequence
  Oligonucleotide
attcagtccatggcaggt

SEQ ID NO: 13 - DNA, 18 bp, Artificial Sequence
  Oligonucleotide
gcgtgtccggttatttct

SEQ ID NO: 14 - DNA, 27 bp, Artificial Sequence
  Oligonucleotide
ccgggaccactcaagcctcgctcccgg

SEQ ID NO: 15 - DNA, 71 bp, Artificial Sequence
  Oligonucleotide
cattcctgaccgttacgacattcgttcacattagttatcgcatttcgggagctaatgaacctgcggcacgt

SEQ ID NO: 16 - DNA, 71 bp, Artificial Sequence
  Oligonucleotide
gcttacagcattgccagtcatttgttcacattagttatcgcatttcgtcgacggggtccaagtaatcgagg

SEQ ID NO: 17 - DNA, 32 bp, Artificial Sequence
  Oligonucleotide
gcagagaatgcgataactaatgtgaactctgc